(19) United States
(12) Patent Application Publication
(10) Pub. No.: US 2023/0141540 A1
(43) Pub. Date: May 11, 2023

(54) A FLOW DEVICE FOR CREATING A FLOW OF BUBBLES AND A VISUALISATION DEVICE WITH A DEVICE FOR CREATING A FLOW OF BUBBLES

(71) Applicant: LiNA Medical International Operations AG, Root (CH)

(72) Inventors: Bjarne WORSOE, Tikøb (DK); Mikkel Haugaard SEIBAEK, Copenhagen N (DK); Jacob Kollerup JENSEN, Hellerup (DK)

(21) Appl. No.: 17/768,141

(22) PCT Filed: Oct. 9, 2020
(86) PCT No.: PCT/EP2020/078446

§ 371 Date: Apr. 11, 2022

(30) Foreign Application Priority Data
Oct. 10, 2019 (EP) 19202389.3

(51) Int. Cl. A61M 31/00; A61M 39/24; A61M 13/00; A61B 1/015; A61B 1/018; A61B 1/05

(52) U.S. Cl. CPC A61M 31/005; A61M 39/24; A61M 13/003; A61B 1/015; A61B 1/018; A61B 1/05; A61M 2210/1433

(57) ABSTRACT

The invention provides a flow device for creating a flow of bubbles of a contrast medium. The device comprises a pump which is configured to provide a flow of the medium and an elongated insertion tube attached to the pump at a proximal end. The elongated insertion tube forms a flow path between the proximal end and an outlet at a distal end.

### FIELD OF THE INVENTION

The present invention relates to a flow device for use in endoscopic procedures, inter alia in hysteroscopy, and particularly to a flow device for creating a flow of bubbles in a patient, e.g. in the uterus, cervix, urethra, or the bladder. The flow device comprises a pump and an elongated insertion tube forming a flow path for the bubbles.

### BACKGROUND OF THE INVENTION

Gynecologists and urologists use various scopes in standard examination procedures, both office-based procedures and in hospital procedures where a scope is inserted e.g. into the uterus for inspection.

For the practitioner, assessment of e.g. fallopian tubal patency includes introduction and observation of a fluid such as air in the uterus. However, the practitioner may experience difficulties obtaining the correct dosing of the contrast medium.

### DESCRIPTION OF THE INVENTION

It is an object of embodiments of the invention to provide an improved flow device for creating a flow of bubbles. It is a further object to facilitate coordination between the release of bubbles and the observation of bubbles.

Accordingly, the invention, in a first aspect, provides a flow device for creating a flow of bubbles of contrast medium, e.g. air. The device comprises a pump, e.g. a manual pump or a motorized pump. The pump is configured to provide a flow of the contrast medium. The flow device further comprises an elongated insertion tube attached to the pump at a proximal end and forming a flow path between the proximal end and an outlet at a distal end.

By providing the flow device with a pump, it is possible to control the flow locally on the device. Compared to manual dosing of air e.g. from an outlet in an operating room, this improves the ability to control and dose the release of the air and thereby more precisely to evaluate tubal patency.

The pump may include a discharge chamber having an elastically compressible wall, e.g. made of a resilient rubber material, e. g. formed with a cup-shape having a bottom part and a wall extending upwards from the bottom part. The bottom part and the wall may particularly have a circular cross-sectional shape. Both the bottom part and the wall may be elastically compressible.

The pump may be operated by manual compression of the wall which thereby reduces the volume of the discharge chamber and presses air contained in the discharge chamber out of the discharge chamber and into the connected elongated insertion tube such that discharged air is pumped through the insertion tube.

The device may comprise a first one-way valve arranged to allow a flow from the discharge chamber into the elongated insertion tube and preventing a flow in the opposite direction.

The contrast medium could be a liquid medium, e.g. colored to create contrast to a distension fluid used for distending the uterus or to provide better reflection of light and thereby increase visibility. Such a colored medium could be delivered to the user together with the device, e.g. contained in the discharge chamber in a sterile condition.

In one embodiment, the contrast medium is air. In this embodiment, the discharge chamber may comprise a communication structure allowing communication of the air with the insertion tube and with the surroundings. In one embodiment, the air is atmospheric air, and the air is communicated from the ambient space into the discharge chamber and from the discharge chamber into the elongated insertion tube.

The communication structure may comprise a valve structure with two independently working one-way valves, one being the above mentioned first one-way valve directed to allow air from the discharge chamber into the elongated insertion tube and preventing air in the opposite direction. The other, second, one-way valve being directed to allow air from the ambient space into the discharge chamber and preventing air in the opposite direction.

The elongated insertion tube defines a space for a certain amount of the contrast medium. Preferably, the volume of the discharge chamber is larger than the space defined by the elongated insertion tube. This allows the discharge chamber to be squeezed one single time to release the contrast medium at the outlet at the distal end and thereby allows the contrast medium to be dispensed by a single operation without necessitating repeated pumping of the discharge chamber.

The discharge chamber may be made from an opaque or semi-transparent material.

For improving hand operation and particularly to allow precise dosage, the resilient compressible wall of the discharge chamber may have an oblong shape extending in the axial direction of the elongated insertion tube. That allows a relatively large enclosed space within the discharge chamber and thereby provides an increased controllability since compression of the wall only has little impact on the volume and therefore provides slow and controllable discharge from the discharge chamber.

The compressible wall may form an elongated tubular element arranged coaxially with the elongated insertion tube, and a flange may be located between the elongated insertion tube and the compressible wall. The flange may be used for connecting the flow device to a control knob of a hysteroscope for controlling rotation of the field of view. The flange thereby forms an interface to the visualization device. For that purpose, the flange may have a shape which allows interlocking between the flange and the control knob for rotation thereof. Such a shape could e.g. be a non-circular shape, or a shape which is non-symmetrical about an axially extending center axis.

When used herein, the terms “radial” and “axial” direction or “radially” and “axially” refers to the center axis through the elongated tube. Radial direction is a direction perpendicular to the center axis and axial direction is in the direction of the center axis.

The flange may have a dimension in the radial direction exceeding the dimension of the compressible wall in the radial direction.

To enable rotation of the flange and thus rotation of the control knob by rotation of the compressible wall, the flange may be rotationally locked to the compressible wall such that the compressible wall can rotate the flange.

The compressible wall may be compressible both in the axial direction and in the radial direction, i.e. transverse to the axial direction thereby allowing operation e.g. by pushing axially with a thump or by squeezing the compressible wall between two fingers. In the above mentioned example of a cup-shaped discharge chamber having a bottom part and a wall extending upwards from the bottom part. The bottom part and the wall may both be elastically compressible thereby allowing the two ways operation by radial and/or axial compression of the discharge chamber.

The distal end of the elongated insertion tube may as an example be inserted through cervix into the uterus of a patient whereby a flow of the contract medium can be provided from the proximal end to the outlet at the distal end where the contrast medium can be released as a flow of bubbles in the uterus. The elongated insertion tube may alternatively be inserted into the bladder for examination hereof.

In the context of the present invention, the term “contrast medium” should be understood as a medium which is visually distinguishable from body tissue, from liquid injected for the purpose of distending the uterus, and/or from body liquids. The bubbles may e.g. be visually distinguishable by use of ultrasound, X-ray, by a camera inserted in uterus, or other suitable techniques. The contrast medium may be selected from different fluids, e.g. a gas, such as air, or a liquid, such as glycerin.

The flow device may thus create a flow of bubbles, where the bubbles are liquid bubbles or gas bubbles. The contrast medium may further have a color which makes it more distinguishable from the surrounding medium.

The pump may comprise a pump being electrically powered. This may be achieved by a battery accommodated in the pump. Alternatively, the pump may be powered via other features of the flow device or other features being operable with the flow device, e.g. a camera. The pump may thus comprise an interface allowing for plug-in of the pump into another part of the flow device or another feature operable with the flow device to thereby power the pump. This may further improve operation of the flow and allow the user to move the device freely without being hindered by cables. Different pumping principles may be applied, e.g. based on movement of a membrane by use of piezo actuators or solenoids etc.

The battery may be elongated and may define an axial direction in which its dimension is larger than in other directions. The battery may be arranged so that the axial direction is transverse to the elongated insertion tube. Consequently, the battery may extend on one or both sides of the elongated insertion tube, whereby the battery or a battery casing for accommodating the battery may form a handle or form part of a handle for the user during operation of the flow device.

The pump may comprise a flow controller configured for releasing single bubbles. Thus, the bubbles may be released one at a time.

Alternatively, or additionally, the pump may comprise a flow controller configured for releasing multiple bubbles.

In one embodiment, the flow controller may be configured for selectively releasing single bubbles or multiple bubbles. Consequently, it may be possible for the user to shift between single bubbles and multiple bubbles, e.g. depending on the examination procedure carried out.

The flow controller may be configured for adjusting a release rate of the bubbles, where the release rate is defined as a specific number of bubbles released per time unit. As an example, the release rate of the bubbles may be adjustable in the range of 2 bubbles per minute to 90 bubbles per minutes, such as 4-70, such as 6-40 bubbles per minute. Dependent on the examination carried out, even more bubbles may be released, such as more than 100, more than 300, and even more than 600 bubbles per minute. The time interval during which the bubbles are released may likewise vary.

The flow controller may read one or both of current and voltage for the pump and based on the reading, the flow controller may determine a counter pressure against the bubble release. Such a counter pressure may e.g. be a result of a distension medium pumped into the uterus or other body cavities for the purpose of distending the uterus or other body parts. Based on the medium, the flow controller may increase or decrease the pumping effect and thus compensate the bubble generation based on the pressure in uterus or other body cavities. In an alternative embodiment, the flow controller may comprise a separate pressure sensor, e.g. a pressure sensor arranged at the insertion tube or forming part of a visualization device used with the flow device. Based on a signal from the pressure sensor, the flow controller may increase or decrease the pumping effect and thus compensate the bubble generation based on the pressure in uterus or other body cavities.

To facilitate control of the flow of the contrast medium, the flow controller may be configured to separate the flow of the contrast medium from the pump into a first flow to the outlet at the distal end of the elongated insertion tube and a second flow to a release located distant from the distal end, whereby only a part of the contrast medium may be released in the form of bubbles at the outlet at the distal end of the insertion tube. As the second flow is a flow from the pump to a release located distant from the distal end, the second flow may be released outside the patient when the elongated insertion tube is arranged with the distal end inside the patient.

The flow controller may be configured to adjust a distribution between the first flow and the second flow. It may thus be possible to increase the first flow and decrease the second flow during operation of the flow device, e.g. if more bubbles are needed in order to carry out the examination of the Fallopian tubes or in relation to other types of examination. Likewise, it may be possible to decrease the first flow while increasing the second flow.

In order to facilitate adjustment of the distribution between the first flow and the second flow, the flow controller may comprise a tube section of an elastically deformable material and may form a second flow path for the second flow. The flow controller may be configured to adjust the distribution by deformation of the tube section. The tube section may be in flow communication with the elongated insertion tube, whereby deformation of the tube section may reduce the second flow and thereby increase the first flow, and vice versa.

A user of the flow device may manually deform the tube section by hand to achieve an accurate and fast adjustment of the distribution e.g. in response to observation of the flow of bubbles from the outlet at the distal end of the elongated insertion tube.

In one embodiment, a clamp may be provided on the tube section, whereby adjustment may be achieved by activation of the clamp.

According to a second aspect, the invention provides a visualization device for visualization of internal tissue. The visualization device may e.g. form a hysteroscope.

The visualization device comprises a control unit, an elongated member, and an image capturing tip, the control unit being dimensioned to be held by a user's hand, the elongated member having a proximal end connected to the control unit and a distal end defining the image capturing tip, wherein the elongated member defines a working channel extending between an inlet in the control unit and an outlet at the image capturing tip, the device further comprising a tool having an elongated tool rod configured for insertion through the working channel.

The control unit may particularly be shaped as a handle for manipulation of the visualization device with one hand. The control unit may particularly be independently powered by a battery, and it may be fitted with different parts such as a monitor, a liquid flushing system, and other parts which are suitable for the procedure. In that way, the device may form a complete, independent, visualization device, e.g. suitable for single use.

The elongated member may be shaped to match the specific purpose. For a hysteroscopic procedure, the elongated member may e.g. be less than 5 mm. in a cross section. Typically, it may have a circular cross section with a diameter of e.g. 4.0-4.8 mm, and a length of approximately 240 mm or more. The elongated member could be made of steel, e.g. with a coating of a low frictional polymer material. The elongated member defines a working channel extending between an inlet at the control unit and an outlet at the image capturing tip. The working channel may allow insertion of a tool through the elongated member, e.g. forceps, a grasper, or a morcellator, or similar tools for taking samples or removing tissue, or a flow device for creating a flow of bubbles.

Thus, the tool may be a flow device for creating a flow of bubbles according to the first aspect of the invention, wherein the tool rod is constituted by the elongated insertion tube.

The skilled person would readily recognize that any feature described in combination with the first aspect of the invention could also be combined with the second aspect of the invention, and vice versa.

The image capturing tip may be constituted by a separate tip-element containing an image capturing structure e.g. in the form of a camera, e.g. a CCD (Charged Coupled Device) and a camera lens. The separate tip-element may include a flow structure for release of air from the image capturing tip and for entering air into the elongated member via the image capturing tip.

The image capturing tip may communicate video signals with a monitor, e.g. a monitor attached to or forming part of the control unit.

In one embodiment, the flow controller configured for releasing bubbles may be controlled via the image capturing tip e.g. via communication with an image recognizing unit. Upon data processing, the data from the image recognizing unit may form basis for the release of bubbles. The image recognizing unit may be a separate unit or may as an example form part of the flow controller or the control unit.

The elongated member or at least a part thereof, e.g. an inner tube within the elongated member, may be rotationally attached to the control unit to allow reorientation of the image capturing tip.

The visualization device may further comprise an illumination structure, e.g. comprising an LED which is housed in the control unit. In that way, heating generated by the LED is at a distance from the patient, and outside the body of the patient. The LED can be powered, e.g. by a battery in the control unit, without having to draw cables through the elongated member. That saves space in the narrow, elongated, member.

The purpose of the illumination structure is to provide illumination suitable for visualization and/or for capturing images internally in the patient. The Kelvin Range: 3500-6000K is suitable e.g. for hysteroscopy. Particularly, a Kelvin Range of 5000-6000K may be suitable and particularly suitable for capturing images of the bubbles.

To facilitate capturing of images from different positions, e.g. different angles without having to move the elongated member, the image capturing tip may be movable relative to the control unit by rotation of a control knob. When inserting the elongated tool rod, e.g. the elongated insertion tube, in the working channel of the visualization device, the elongated tool may at least partly cover the control knob thereby at least partly hindering rotation of the control knob. This may be overcome by the tool itself, as the tool may form an interface configured to engage the control knob to allow rotation of the control knob by rotation of the tool while the elongated tool rod is inserted in the working channel. Thus, the flow device according to the first aspect of the invention may from an interface configured to engage the control knob and particularly allowing rotation of the control knob.

To facilitate engagement between the tool, i.e. e.g. a flow device, and the visualization device, the interface of the tool and the control knob may define geometrically interlocking features. I.e. the interlocking features of the tool may be of a shape which matches a shape of the interlocking feature of the control knob to thereby form a geometrical locking between these features. In one embodiment, the interlocking feature of the tool may be in the form of a protrusion being insertable into an interlocking feature of the control knob in the form of an indentation or opening.

Alternatively, engagement between the tool, e.g. a flow device, and the visualization device may be achieved by friction, by magnetism, or by other suitable means.

Furthermore, engagement may in one embodiment ensure a firm locking between the tool, e.g. a flow device, and the visualization device, whereas the engagement in an alternative embodiment may result in a limitation of the relative movement of the tool, e.g. a flow device, and the visualization device without a firm locking. As an example, the tool and the visualization device may rotate up to 30 degrees relative to each other when engaged.

In one embodiment, the compressible wall is rotationally locked to that part of the flow device which engages the control knob. That allows the control knob to be rotated by the tool by rotation of the compressible wall and that allows compression and thus bubble generation simultaneously with the rotation, and by use of one hand.

The tool rod may be longer than the elongated member, and particularly, it may have a length allowing it to extend axially ahead of the image capturing tip in the distal direction.

That may allow the image capturing tip to capture activities at the tip of the tool rod, e.g. release of bubbles from the distal outlet of the flow device. In case of a flow device, the outlet at the distal end may particularly be at a distance from the image capturing tip, i.e. ahead of the image capturing tip in the distal direction sufficient for the outlet to be located where the image capturing tip can establish a clear focus. This may particularly be 4-20 mm. such as 6-16 mm. in front of the image capturing tip.

According to a third aspect, the invention provides a method for detecting Fallopian tubal patency by use of a flow device according to the first aspect of the invention or a visualization device according to the second aspect of the invention, the method comprising the steps of:

inserting the elongated tube through cervix into the uterus of a patient,
operating the pump to provide a flow of at least one bubble of the contrast medium at the outlet, and
detecting a flow direction of the at least one bubble.

The method may comprise a step of inserting the elongated member through cervix into the uterus of the patient, and subsequently inserting the elongated tube via the working channel. The flow direction may be detected by use of the image capturing tip.

A skilled person would readily recognize that any feature described in combination with the first aspect and/or second aspect of the invention could also be combined with the third aspect of the invention, and vice versa.

The flow device according to the first aspect and the visualization device according to the second aspect of the invention are very suitable for performing the method steps according to the third aspect of the invention. The remarks set forth above in relation to the devices are therefore equally applicable in relation to the method.

### BRIEF DESCRIPTION OF THE DRAWINGS

Embodiments of the invention will now be further described with reference to the drawings, in which:

FIG. 1 illustrates an embodiment of a flow device according to the invention where the elongated insertion tube is inserted into uterus;

FIG. 2 illustrates parts of an embodiment of a flow device according to the invention;

FIG. 3 illustrates a sideview of a flow device according to the invention;

FIG. 4 illustrates an enlarged view of a flange and a compressible wall;

FIG. 5 illustrates an exploded view of the flow device in FIGS. 3 and 4;

FIGS. 6-8 illustrate a function of a set of one-way valves and the compressible wall;

FIGS. 9-10 illustrate interaction between the flow device and a visualization device;

FIG. 11 illustrates insertion of an embodiment of a flow device according to the invention into a visualization device according to the invention; and

FIG. 12 illustrates operation of an embodiment of a flow device according to the invention while inserted into a visualization device according to the invention.

### DETAILED DESCRIPTION OF THE DRAWINGS

The detailed description and specific examples, while indicating embodiments of the invention, are given by way of illustration only, since various changes and modifications within the spirit and scope of the invention will become apparent to those skilled in the art from this detailed description.

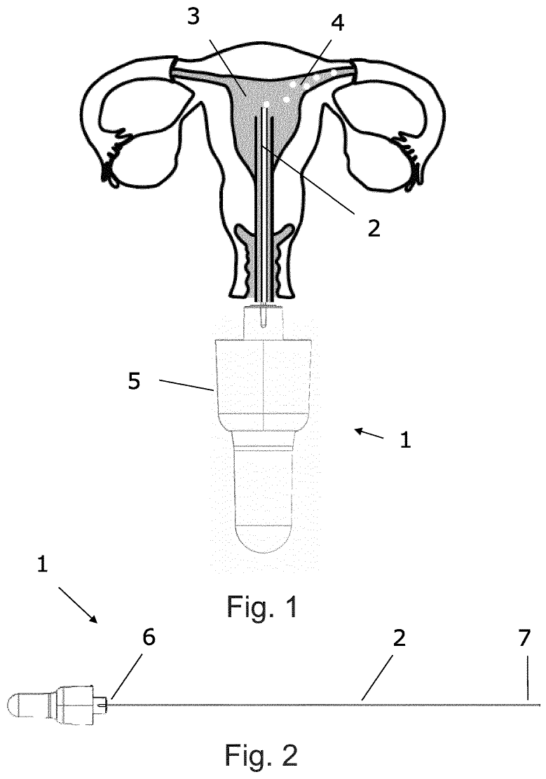

FIGS. 1 and 2 illustrate parts of an embodiment of a flow device 1 according to the invention where the elongated insertion tube 2 is inserted into uterus 3. The flow device 1 is configured for creating a flow of bubbles 4 of a contrast medium. The illustrated device is configured for use of atmospheric air as contrast medium. The flow device 1 comprises a pump 5 configured to provide a flow of the air and an elongated insertion tube 2 attached to the pump 5 at a proximal end 6 and forming a flow path between the proximal end and an outlet at a distal end 7.

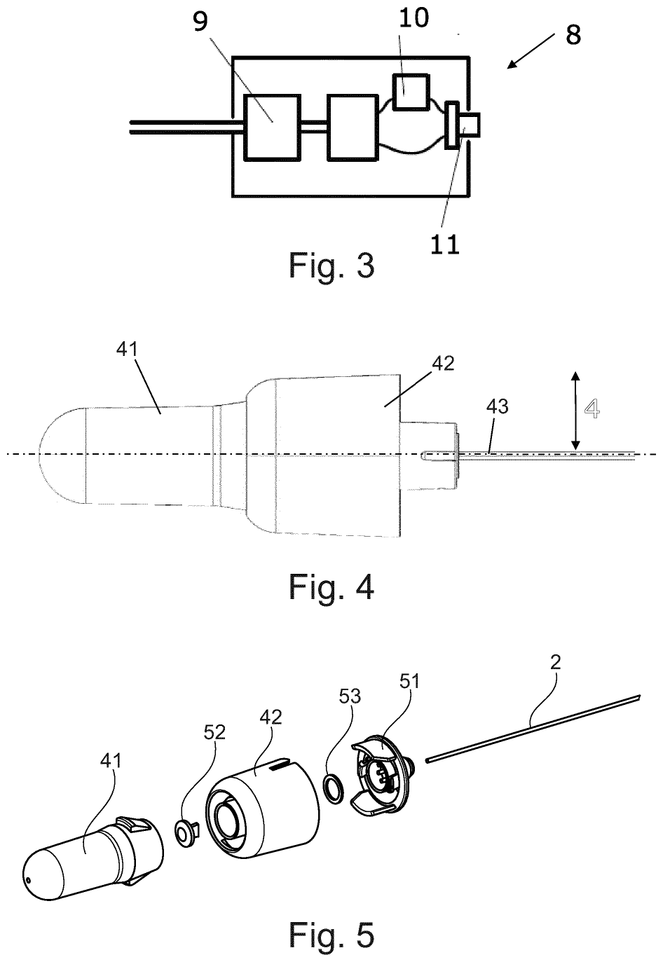

In the illustrated embodiment, the pump 5 is an air pump. FIG. 3 illustrates an alternative pump in the form of a powered pump 8 having a controlled release valve 9 and a flow controller 10 for configured for releasing single bubbles and/or multiple bubbles.

In the illustrated embodiment, the flow controller 10 is operated via the knob 11 arranged at the proximal end of the pump 8.

FIG. 4 illustrates the manually operated pump from FIGS. 1 and 2 in an enlarged view. The pump comprises a discharge chamber formed by a resilient compressible wall 41 and a flange 42. The compressible wall 41 is cup shaped and forms a side wall and a bottom forming a curvature extending into the sidewall to provide a torpedo-shaped end of the compressible wall. Both the sidewall and the bottom are made from an elastically deformable material, and the pump can therefore be actuated by an axially and/or a radially directed pressure against the sidewall and/or the bottom.

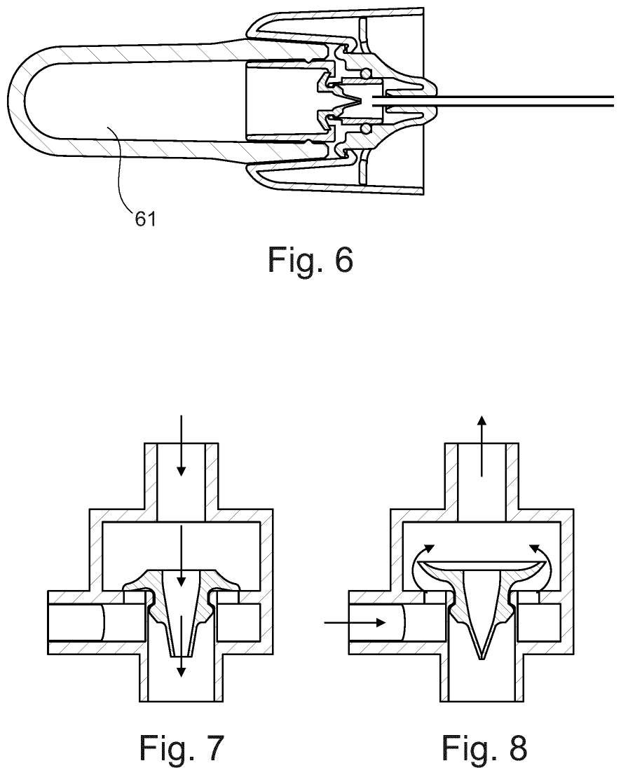

The discharge chamber receives air from the surroundings via a first one-way valve and to delivers the air through the elongated insertion tube via a second one-way valve which is illustrated in further details in FIGS. 6-8.

The compressible wall forms an elongated tubular element arranged coaxially with the elongated insertion tube illustrated by the common axis 43.

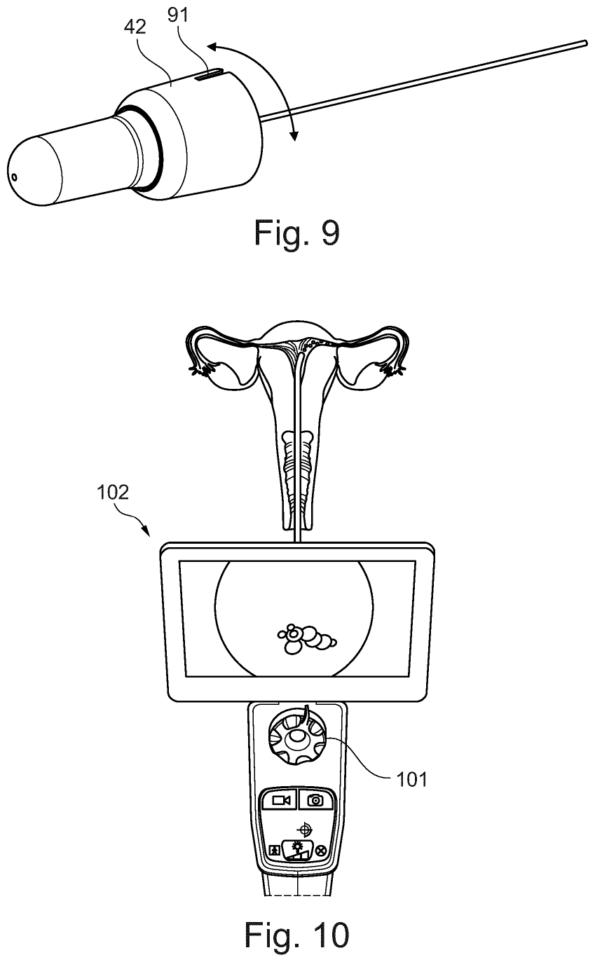

The flange 42 is located between the elongated insertion tube and the compressible wall and has a shape which is non-symmetric or non-circular about an axially extending center axis. The asymmetric shape is shown in FIG. 9 and is defined by an incision 91 matching a projection of a control knob of a visualization device.

The flange has radial dimension illustrated by the arrow 44 which exceeds the dimension of the compressible wall in the radial direction.

The flange is rotationally locked to the compressible wall such that the compressible wall can rotate the flange and thereby rotate the control knob of the visualization device.

FIG. 5 illustrates an exploded view of the visualization device, and particularly shows that the pump comprises an end closure 51, the flange 42, the compressible wall 41 and a valve structure including a one-way valve 52 and an O-ring 53 sealing between the valve structure and the elongated insertion tube.

FIGS. 6-8 illustrate details of the two one-way valves included in the valve structure. FIG. 1 illustrates a step 1 in which the compressible wall is squeezed and air flows through the elongated insertion tube. In FIG. 9, the compressible wall is released and air flows from outside into the discharge chamber 61.

FIGS. 9-10 illustrate the details of the non-symmetric shape of the flange 42 constituted by an incision 91 engaging a projection on the control knob 101 of the visualization device 102.

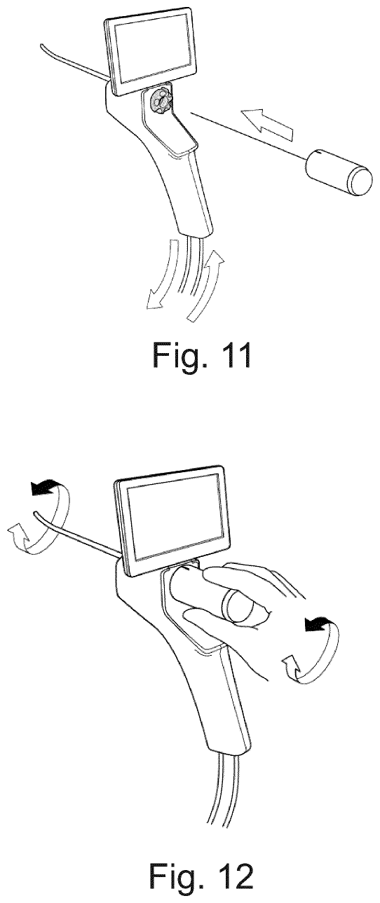

FIG. 11 illustrates the flow device 1 when inserted into the visualization device 102. The visualization device 102 is configured for visualization of internal tissue and comprises a control unit 111, an elongated member 112, and an image capturing tip 113.

The control unit 111 is dimensioned to be held by a user's hand. The elongated member 112 has a proximal end 114 connected to the control unit 111 and a distal end 115 defining the image capturing tip.

The elongated member 112 defines a working channel extending between an inlet in the control unit 111 and an outlet at the image capturing tip 113.

The visualization device 102 further comprises the flow device 1 being inserted through an opening 116 in the control unit 111 and into the working channel formed by the elongated member 112.

In the illustrated embodiment, the visualization device 102 comprises a display unit for configured to display electronic video signals from the image capturing tip 113.

To facilitate capturing of images from different angles without having to move the elongated member 112, the image capturing tip 113 is movable relative to the control unit 111 by rotation of a control knob 101. The flow device 1 forms an interface (not shown) which is configured to engage the control knob 101 to allow rotation of the control knob 101 by rotation of the flow device 1 while the elongated insertion tube 2 is inserted in the working channel of the elongated member 112.

FIG. 12 illustrates operation of the flow device 1 while inserted into a visualization device 102. By the arrows, it is illustrated that rotation of the pump 5 of the flow device 1 will cause the image capturing tip 113 to rotate due to engagement between the flow device 1 and the control knob 101 of the visualization device 102.

### LISTED EMBODIMENTS

1. A flow device for creating a flow of bubbles of a contrast medium, the device comprising a motorized pump unit configured to provide a flow of the contrast medium and an elongated insertion tube attached to the pump unit at a proximal end and forming a flow path between the proximal end and an outlet at a distal end.

2. A flow device according to embodiment 1, wherein the pump unit comprises a pump being electrically powered.

3. A flow device according to embodiment 2, wherein the pump unit is powered by a battery, and wherein the battery is elongated and defines an axial direction being transverse to the elongated insertion tube.

4. A flow device according to any of the preceding embodiments, wherein the pump unit comprises an air pump.

5. A flow device according to any of the preceding embodiments, wherein the pump unit comprises a flow controller configured for releasing single bubbles.

6. A flow device according to any of the preceding embodiments, wherein the pump unit comprises a flow controller configured for releasing multiple bubbles.

7. A flow device according to embodiment 5 or 6, wherein the flow controller is configured for selectively releasing single bubbles or multiple bubbles.

8. A flow device according to any of embodiments 5-7, wherein the flow controller is configured for adjusting a release rate of the bubbles.

9. A flow device according to any of embodiments 5-8, wherein the flow controller is configured to separate the flow of the contrast medium from the pump unit into a first flow to the outlet and a second flow to a release located distant from the distal end.

10. A flow device according to embodiment 9, wherein the flow controller is configured to adjust a distribution between the first flow and the second flow.

11. A flow device according to any of embodiments 5-10, wherein the flow controller is configured to increase or decrease a pumping effect and thus compensate the bubble generation based on a pressure a body cavity.

12. A flow device according to embodiment 10 or 11, wherein the flow controller comprises a tube section of an elastically deformable material and forming a second flow path for the second flow and being configured to adjust the distribution by deformation.

13. A visualization device for visualization of internal tissue, the device comprising a control unit, an elongated member, and an image capturing tip, the control unit being dimensioned to be held by a user's hand, the elongated member having a proximal end connected to the control unit and a distal end defining the image capturing tip, wherein the elongated member defines a working channel extending between an inlet in the control unit and an outlet at the image capturing tip, the device further comprising a tool having an elongated tool rod configured for insertion through the working channel.

14. A visualization device according to embodiment 13, wherein the tool is a flow device for creating a flow of bubbles according to any of embodiments 1-12, and wherein the tool rod is constituted by the elongated tube.

15. A visualization device according to embodiment 13 or 14, wherein the image capturing tip is movable relative to the control unit by rotation of a control knob, and wherein the tool forms an interface configured to engage the control knob to allow rotation of the control knob by rotation of the tool while the elongated tool rod inserted in the working channel.

16. A visualization device according to embodiment 15, wherein the interface and the control knob defines geometrically interlocking features.

17. A method for detecting Fallopian tubal patency by use of a flow device according to any of embodiments 1-12 or a visualization device according to any of embodiments 14-16, the method comprising the steps of:

inserting the elongated tube through cervix into the uterus of a patient
operating the pump unit to provide a flow of at least one bubble of the contrast medium at the outlet, and
detecting a flow direction of the at least one bubble.

18. A method according to embodiment 17 comprising a step of inserting the elongated member through cervix into the uterus of the patient, and subsequently inserting the elongated tube via the working channel, and wherein the flow direction is detected by use of the image capturing tip.

1. A flow device for creating a flow of bubbles of a contrast medium, the device comprising a pump configured to provide a flow of the contrast medium and an elongated insertion tube extending axially from a proximal end to a distal end and forming a flow path between the pump in the proximal end and an outlet at the distal end.

2. A flow device according to claim 1, wherein the pump is manually operated.

3. The flow device according to claim 2, wherein the pump comprises a discharge chamber having a resilient compressible wall.

4. The flow device according to claim 3, comprising a first one-way valve directed to allow a flow of the contrast medium from the discharge chamber into the elongated insertion tube and preventing flow in the opposite direction.

5. The flow device according to claim 1 and configured for air as contrast medium, the discharger chamber being arranged for receiving the air from the surroundings via a second one-way valve directed to allow a flow of the air from an ambient space into the discharge chamber and preventing a flow in the opposite direction.

6. The flow device according to claim 3, wherein the compressible wall has an elongated tubular shape arranged coaxially with the elongated insertion tube.

7. The flow device according to claim 3, comprising a flange located between the elongated insertion tube and the compressible wall, the flange having a shape allowing rotationally interlocking with a control knob of a visualization device.

8. The flow device according to claim 7, wherein the flange has a dimension in a radial direction being transverse to the axial direction and exceeding the dimension of the compressible wall in the radial direction.

9. The flow device according to claim 7, wherein the flange is rotationally locked to the compressible wall such that the compressible wall can rotate the flange.

10. The flow device according to claim 3, wherein the elongated insertion tube defines a space for a certain amount of the contrast medium, and the discharge chamber forms space for a larger amount of the contrast medium.

11. The flow device according to claim 3, wherein the discharge chamber is made from an opaque material.

12. The flow device according to claim 3, wherein the compressible wall is compressible both in the axial direction and in a radial direction transverse to the axial direction.

13. A visualization device for visualization of internal tissue, the device comprising a control unit, an elongated member, and an image capturing tip, the control unit being dimensioned to be held by a user's hand, the elongated member having a proximal end connected to the control unit and a distal end defining the image capturing tip, wherein the elongated member defines a working channel extending between an inlet in the control unit and an outlet at the image capturing tip, the device further comprising a tool having an elongated tool rod configured for insertion through the working channel.

14. The visualization device according to claim 13, wherein the tool is a flow device for creating a flow of bubbles of a contrast medium, the device comprises a pump configured to provide a flow of the contrast medium and an elongated insertion tube extending axially from a proximal end to a distal end and forming a flow path between the pump in the proximal end and an outlet at the distal end, and wherein the tool rod is constituted by the elongated tube.

15. The visualization device according to claim 13, wherein the image capturing tip is movable relative to the control unit by rotation of a control knob, and wherein the tool forms an interface configured to engage the control knob to allow rotation of the control knob by rotation of the tool while the elongated tool rod is inserted in the working channel.

16. The visualization device according to claim 15, wherein the interface and the control knob defines geometrically interlocking features.

17. The visualization device according to claim 14, wherein the rotation of the control knob by rotation of the tool can be carried out by rotation of the compressible wall.

18. The visualization device according to claim 13, wherein the tool rod is longer than the elongated member.

19. The method for detecting Fallopian tubal patency by use of a flow device according to claim 1 or a visualization device-for visualization of internal tissue, the visualization device comprising a control unit, an elongated member, and an image capturing tip, the control unit being dimensioned to be held by a user's hand, the elongated member having a proximal end connected to the control unit and a distal end defining the image capturing tip, wherein the elongated member defines a working channel extending between an inlet in the control unit and an outlet at the image capturing tip, the visualization device further comprising a tool having an elongated tool rod configured for insertion through the working channel, the method comprising the steps of:
inserting the elongated tube through cervix into the uterus of a patient
operating the pump to provide a flow of at least one bubble of the contrast medium at the outlet, and
detecting a flow direction of the at least one bubble.

20. The method according to claim 19 comprising a step of inserting the elongated member through cervix into the uterus of the patient, and subsequently inserting the elongated tube via the working channel, and wherein the flow direction is detected by use of the image capturing tip.
